(19) United States
(12) Patent Application Publication
(10) Pub. No.: US 2023/0293412 A1
(43) Pub. Date: Sep. 21, 2023

(54) SHAMPOOS AND METHODS OF PREPARING AND USING THE SAME

(71) Applicant: SHANDONG BENZHEN COSMETICS CO., LTD, Shandong (CN)

(72) Inventors: Zhengmei HUANG, Shandong (CN); Jinping DING, Shandong (CN); Hui LIN, Shandong (CN)

(73) Assignee: SHANDONG BENZHEN COSMETICS CO., LTD, Shandong (CN)

(21) Appl. No.: 18/017,597

(22) PCT Filed: Jul. 27, 2020
(86) PCT No.: PCT/CN2020/104853

§ 371 Date: Jan. 23, 2023

(51) Int. Cl. A61K 8/44; A61Q 5/02; A61K 8/73; A61K 8/04; A61K 8/96

(52) U.S. Cl. CPC A61K 8/44; A61Q 5/02; A61K 8/73; A61K 8/042; A61K 8/732; A61K 8/965; A61K 2800/56; A61K 2800/33; A61K 2800/30; A61K 2800/524

(57) ABSTRACT

It relates to shampoos and kits or packages comprising the same. It also relates to methods of preparing and using the shampoos.

### TECHNICAL FIELD

The present disclosure relates to shampoos and kits or packages comprising the same. Methods of preparing and using the shampoos are also provided.

### BACKGROUND

In the following discussion, certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Current shampoos contain various ingredients, and the combined effect of these ingredients can cleanse the scalp and hair. Usually the main cleansing ingredient in the shampoo is the surfactant. When shampoo is mixed with water, foam can be generated, but the amount of foam does not reflect the strength of the cleaning ability.

Surfactants used in current shampoo mainly include sodium lauryl alcohol polyether ester sulfate (AES), coconut oil diethanolamide (6501), Coco-amide propyl betaine (CAB), quaternary ammonium salt softener, and some functional additives, such as silicone smoothing brightener, anti-dandruff agent, pearlescent agent, preservative, essence, pigment, etc. However, the surfactants used in the existing shampoos are more or less harmful to the human body and are not environmentally friendly.

There are more and more people with gray hair and hair loss, and they are getting younger and younger, which may be due to factors such as work pressure, staying up late and even uneven nutrition. But this is also true for many people with regular and healthy lives. The more developed countries have more serious hair problems. Before the 1990s in China, there were very few hair loss problems and gray hair problems. But since the beginning of this century, hair problems have been increasing rapidly. According to statistics from online shopping, young people in their 20s have a rapid growth in hair loss shampoos. Large-scale statistics also show that the more people who use shampoo, the more serious the hair problem, especially the people who shampoo every day. The main reason is that all current shampoos use a lot of chemically synthesized substances, especially the surfactant(s). For many active agents in current shampoos, they have a very strong ability to decontaminate, excessively remove oil, which destroys the oil barrier of the scalp. Not only that, surfactant is a skin transdermal absorption accelerator that can penetrate into the hair follicle and enter the secretory ducts of the sweat glands and oil glands. It can also penetrate the cuticle into the skin and even into the blood. Washing hair every day will keep deposit these chemical substances into the subcutaneous, which accumulate over a long period continuously, attack or destroy the hair follicle dermal papilla of hair and the skin melanin cell survival environment. The accumulation of such chemical substances eventually leads to hair loss and/or premature gray hair. Surfactant and other chemical material can also destroy cells.

In summary, traditional shampoo products use fungicides, surfactants (including anions, cations and non-ions) and other chemical synthetic substances, which will damage the skin microecology, damage the skin barrier, and enter the skin. This is the main reason for the widespread occurrence of modern human hair problems.

There is a growing need for improved shampoo products that are effective in cleaning hair, but less toxic or damaging to the hair, skin and skin microecology. The present disclosure addresses this and other related needs.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In one aspect, provided herein is a shampoo, when diluted for at least about 80 times, the surface tension of the diluent from the shampoo, measured at an ambient temperature, is at about 35 mN/m or higher.

In another aspect, provided herein is a kit or package, which kit or package comprises the above shampoo, and an instruction for washing a subject's hair using the shampoo.

In still another aspect, provided herein are methods for washing a subject's hair, which methods comprise contacting a subject's hair with the above shampoo.

### BRIEF DESCRIPTION OF THE DRAWINGS

FIG. 1 illustrates effects of exemplary shampoo XF1-12 on Volunteer 1 (42 years old, female).

FIG. 2 illustrates effects of exemplary shampoo XF1-12 on Volunteer 2 (9 years old, female).

FIG. 3 illustrates effects of exemplary shampoo XF1-6 on Volunteer 3 (41 years old, male).

FIG. 4 illustrates effects of exemplary shampoo XF1-6 on Volunteer 4 (53 years old, male).

FIG. 5 illustrates effects of exemplary shampoo XF1-6 on Volunteer 5 (48 years old, male).

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### A. Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.” As used herein, the singular forms “a,” “an,” and “the” include the plural reference unless the context clearly dictates otherwise.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “average” as used herein refers to either a mean or a median, or any value used to approximate the mean or the median, unless the context clearly indicates otherwise.

As used herein the term “sample” refers to anything which may contain a target molecule for which analysis is desired, including a biological sample. As used herein, a “biological sample” can refer to any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine, sweat, semen, stool, sputum, tears, mucus, amniotic fluid or the like, an effusion, a bone marrow sample, ascitic fluid, pelvic wash fluid, pleural fluid, spinal fluid, lymph, ocular fluid, extract of nasal, throat or genital swab, cell suspension from digested tissue, or extract of fecal material, and tissue and organ samples from animals and plants and processed samples derived therefrom.

As used herein, “disease or disorder” refers to a pathological condition in an organism resulting from, e.g., infection or genetic defect, and characterized by identifiable symptoms.

As used herein, a “preservative” refers to a substance or a chemical that is added to a shampoo to reduce or prevent damage, decomposition or other undesirable effects by microbial growth and/or metabolism, or by an undesirable chemical reaction. As used herein, an “antimicrobial preservative” refers to a substance or a chemical that is added to a shampoo to reduce or prevent damage, decomposition or other undesirable effects by microbial growth and/or metabolism.

As used herein, a “surfactant” refers to a substance or a chemical that lowers the surface tension (or interfacial tension) between two liquids, between a gas and a liquid, or between a liquid and a solid. A surfactant is usually an organic compound that is amphiphilic, meaning it contains both a hydrophobic group and a hydrophilic group. Therefore, a surfactant contains both a water-insoluble (or oil-soluble) component and a water-soluble component. A surfactants may diffuse in water and absorbs at interface between air and water or at the interface between oil and water, in the case where water is mixed with oil. The water-insoluble hydrophobic group may extend out of the bulk water phase, into the air or into the oil phase, while the water-soluble head group remains in the water phase.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, a subject refers to an animal, a non-human mammal or a human. As used herein, “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee.

Other objects, advantages and features of the present invention will become apparent from the following specification taken in conjunction with the accompanying drawings.

### B. Shampoos and Related Kits, Packages and Articles of Manufacture

In one aspect, provided herein is a shampoo, when diluted for at least about 80 times, the surface tension of the diluent from the shampoo, measured at an ambient temperature, is at about 35 mN/m or higher.

In some embodiments, when diluted for about 80 times, the surface tension of the diluent from the shampoo, measured at an ambient temperature, is at about 35 mN/m or higher. In some embodiments, diluted for about 80 times, the surface tension of the diluent from the shampoo, measured at an ambient temperature, ranges from about 35 mN/m to about 68 mN/m, e.g., at about 35 mN/m, 40 mN/m, 45 mN/m, 50 mN/m, 55 mN/m, 60 mN/m, 65 mN/m, 68 mN/m, or any suitable subrange thereof. The surface tension of the diluent from the shampoo can be measured at any suitable ambient temperature. In some embodiments, the ambient temperature can range from about 22° C. to about 26° C., e.g., at about 22° C., 23° C., 24° C., 25° C., or 26° C., or any suitable subrange thereof.

In some embodiments, the present shampoo does not comprise a surfactant, or at least does not comprise a surfactant at a detectable level.

In some embodiments, the present shampoo can comprise a surfactant. The present shampoo can comprise any suitable surfactant. For example, the present shampoo can comprise a surfactant which, as an ingredient before incorporated into the shampoo and diluted for at least about 6,000 times, the surface tension of the diluent from the surfactant is at about 45 mN/m or higher. For example, the present shampoo can comprise any suitable surfactant that is an anionic surfactant, a zwitterionic surfactant, a nonionic surfactant, or a mild surfactant, e.g., a mild amino acid surfactant. In some embodiments, the present shampoo can comprise a surfactant that is sodium lauroyl sarcosinate, methyl glucose dioleate (DOE-120), ASB-14 (amidosulfobetaine-14), or Alkyl glycoside (C8-C10) (APG1). In some preferred embodiments, the present shampoo can comprise a surfactant that is sodium lauroyl sarcosinate.

The present shampoo can comprise any suitable level or concentration of the surfactant. For example, the present shampoo can comprise the surfactant at a level or concentration ranging from about 0.5% (w/w) to about 5% (w/w), e.g., at about 0.5% (w/w), 1% (w/w), 2% (w/w), 3% (w/w), 4% (w/w), 5% (w/w), or any suitable subrange thereof. In some embodiments, the present shampoo can comprise sodium lauroyl sarcosinate at a level or concentration ranging from about 0.5% (w/w) to about 5% (w/w), e.g., at about 0.5% (w/w), 1% (w/w), 2% (w/w), 3% (w/w), 4% (w/w), 5% (w/w), or any suitable subrange thereof. In some embodiments, the present shampoo can comprise a sodium lauroyl sarcosinate composition comprising about 30% (w/w) sodium lauroyl sarcosinate at a level or concentration ranging from about 1.5% (w/w) to about 15% (w/w), e.g., at about 1.5% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), or any suitable subrange thereof.

In some embodiments, the present shampoo can further comprise any suitable gel-forming natural polymer. In the present shampoo, a gel formed by the natural polymer can have any suitable particle size or diameter. For example, in the present shampoo, a gel formed by the natural polymer can have a particle size or diameter ranging from about 50 μm to about 5 mm, e.g., at about 50 μm, 100 μm, 200 μm, 300 μm, 400 μm, 500 μm, 600 μm, 700 μm, 800 μm, 1 mm, 2 mm, 3 mm, 4 mm, 5 mm, or any suitable subrange thereof. In some embodiments, in the present shampoo, the gel formed by the natural polymer has a particle size or diameter ranging from about 100 μm to about 3 mm.

In another example, in the present shampoo, the gel formed by the natural polymer can encapsulate the surfactant. In still another example, in the present shampoo, the natural polymer can have reduced or minimized deposition level on a subject's hair.

The natural polymer used in the present shampoo can be obtained or derived from any suitable sources. For example, the natural polymer used in the present shampoo can be obtained or derived from a plant, a marine algae, a microorganism or an animal.

The present shampoo can comprise any suitable gel-forming natural polymer. For example, the natural polymer used in the present shampoo can be agar, carrageenan, curd lan, or gellan gum.

The present shampoo can comprise any suitable level or concentration of the gel-forming natural polymer. For example, in the present shampoo, the level or concentration of the natural polymer can range from about 0.1% (w/w) to about 10% (w/w), e.g., at about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), or any suitable subrange thereof.

In some embodiments, the present shampoo can further comprise any suitable humectant. For example, the present shampoo can comprise any suitable humectant that is glycerin, sorbitol, maltose syrup, an isomalt oligosaccharide or a small-molecule humectant. The present shampoo can comprise any suitable level or concentration of the humectant. For example, in the present shampoo, the level or concentration of the humectant ranges from about 5% (w/w) to about 75% (w/w), e.g., at about 5% (w/w), 10% (w/w), 15% (w/w), 20% (w/w), 25% (w/w), 30% (w/w), 35% (w/w), 40% (w/w), 45% (w/w), 50% (w/w), 55% (w/w), 60% (w/w), 65% (w/w), 70% (w/w), 75% (w/w), or any suitable subrange thereof.

In some embodiments, the present shampoo does not comprise a preservative, or at least does not comprise a preservative at a detectable level.

In some embodiments, the present shampoo can further comprise a preservative. The present shampoo can comprise any suitable preservative. For example, the present shampoo can comprise a preservative that is selected from the consisting of isothiazolinone (isothiazolone or Casson), 1,2-hexanediol, caprylyl glycol, ethylhexylglycerin, phenoxyethanol, ethylhexylglycerin, propanediol and hydroxyacetophenone. The present shampoo can comprise any suitable level or concentration of the preservative. For example, in the present shampoo, the level or concentration of the preservative can range from 0.01% (w/w) to about 5% (w/w), e.g., at about 0.01% (w/w), 0.05% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), or any suitable subrange thereof.

In some embodiments, the present shampoo can further comprise any suitable additional substance. For example, the additional substance can be starch, cellulose, cyclodextrin, natural sea mud and/or diatomaceous earth. The present shampoo can comprise any suitable level or concentration of the additional substance. For example, in the present shampoo, the level or concentration of the additional substance can range from about 1% (w/w) to about 50% (w/w), e.g., at about 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w), or any suitable subrange thereof.

In some embodiments, the present shampoo can further comprise any suitable water. For example, the present shampoo can further comprise distilled water, e.g., triple distilled water. The present shampoo can comprise any suitable level or concentration of the water. For example, in the present shampoo, the level or concentration of the water can range from 10% (w/w) to about 90% (w/w), e.g., at about 10% (w/w), 15% (w/w), 20% (w/w), 25% (w/w), 30% (w/w), 35% (w/w), 40% (w/w), 45% (w/w), 50% (w/w), 55% (w/w), 60% (w/w), 65% (w/w), 70% (w/w), 75% (w/w), 80% (w/w), 85% (w/w), 90% (w/w), or any suitable subrange thereof.

The present shampoo can be configured for any suitable form. For example, the present shampoo can be configured as a solid, e.g., a bar, a semi-liquid, a flowing liquid, a viscos liquid, a gel, a flowing gel, a soft gel or a liquid.

In some embodiments, the present shampoo can be configured as a flowing gel and can comprise a natural polymer, e.g., agar, a surfactant, e.g., sodium lauroyl sarcosinate, a preservative, e.g., Casson, and water. The present shampoo can further comprise a humectant, e.g., glycerin, sorbitol, maltose syrup, an isomalt oligosaccharide or a small-molecule humectant. In the present shampoo, the level or concentration of the natural polymer can range from 0.1% (w/w) to about 10% (w/w), the level or concentration of the surfactant can range from 0.8% (w/w) to about 4% (w/w), the level or concentration of the preservative can range from 0.01% (w/w) to about 5% (w/w), the level or concentration of the water can range from 50% (w/w) to about 85% (w/w), and the level or concentration of the humectant can range from 5% (w/w) to about 15% (w/w).

In some embodiments, the present shampoo can be configured as a flowing gel and comprises a natural polymer, e.g., agar, a surfactant, e.g., sodium lauroyl sarcosinate, and water, and does not comprise a preservative. The present shampoo can further comprise a humectant, e.g., glycerin, sorbitol, maltose syrup, an isomalt oligosaccharide or a small-molecule humectant. In the present shampoo, the level or concentration of the natural polymer can range from 0.1% (w/w) to about 6% (w/w), the level or concentration of the surfactant can range from 2% (w/w) to about 4% (w/w), the level or concentration of the water can range from 15% (w/w) to about 40% (w/w), and the level or concentration of the humectant can range from 30% (w/w) to about 70% (w/w).

In some embodiments, the present shampoo can be configured as a solid gel and comprises a natural polymer, e.g., agar, a surfactant, e.g., sodium lauroyl sarcosinate, a preservative, e.g., Casson, and water. The present shampoo can further comprise a humectant, e.g., glycerin, sorbitol, maltose syrup, an isomalt oligosaccharide or a small-molecule humectant. In the present shampoo, the level or concentration of the natural polymer can range from 1% (w/w) to about 3% (w/w), the level or concentration of the surfactant can range from 2% (w/w) to about 4% (w/w), the level or concentration of the preservative can range from 0.01% (w/w) to about 5% (w/w), the level or concentration of the water can range from 40% (w/w) to about 80% (w/w), and the level or concentration of the humectant can range from 5% (w/w) to about 15% (w/w).

In some embodiments, the present shampoo can be configured as a solid gel and comprises a natural polymer, e.g., agar, a surfactant, e.g., sodium lauroyl sarcosinate, and water, and does not comprise a preservative. The present shampoo can further comprise a humectant, e.g., glycerin, sorbitol, maltose syrup, an isomalt oligosaccharide or a small-molecule humectant. In the present shampoo, the level or concentration of the natural polymer can range from 0.5% (w/w) to about 3% (w/w), the level or concentration of the surfactant can range from 1% (w/w) to about 4% (w/w), the level or concentration of the water can range from 20% (w/w) to about 45% (w/w), and the level or concentration of the humectant can range from 40% (w/w) to about 75% (w/w).

In some embodiments, the present shampoo can be configured as a flowing gel and comprises a natural polymer, e.g., agar, a preservative, e.g., Casson, and water, and does not comprise a surfactant. The present shampoo can further comprise a humectant, e.g., glycerin, and one or more substances selected from Psyllium Husk Powder, Konjac Glucomannan, Betaine, corn starch, and Red mud. In the present shampoo, the level or concentration of the natural polymer can range from 0.1% (w/w) to about 10% (w/w), the level or concentration of the water can range from 40% (w/w) to about 70% (w/w), and the level or concentration of the humectant can range from 5% (w/w) to about 10% (w/w), the level or concentration of the preservative can range from 0.01% (w/w) to about 5% (w/w), the level or concentration of the Psyllium Husk Powder can range from 0.1% (w/w) to about 2% (w/w), the level or concentration of the Konjac Glucomannan can range from 0.5% (w/w) to about 1.5% (w/w), the level or concentration of the Betaine can range from 5% (w/w) to about 15% (w/w), the level or concentration of the corn starch can range from 10% (w/w) to about 30% (w/w), and the level or concentration of the Red mud can range from 1% (w/w) to about 30% (w/w).

The present shampoo can be configured for any suitable uses. For example, the present shampoo can be configured as a baby shampoo.

In another example, the present shampoo can be configured as a shampoo for washing hair of a subject or a human with a skin condition that affects the scalp of the subject or human. The skin condition can be Dandruff or Seborrhoeic dermatitis (or seborrhoea). In some embodiments, the present shampoo can further comprise an ingredient for treating the skin condition, Dandruff or Seborrhoeic dermatitis (or seborrhoea). Any suitable ingredient can be used. For example, the ingredient can be an antifungal agent, e.g., ketoconazole, zinc pyrithione (or pyrithione zinc), or selenium disulfide (or selenium sulfide).

In still another example, the present shampoo can be configured as an animal shampoo. The present shampoo can further comprise an insecticide, or a medication for treating a skin condition or parasite infestation. Any suitable medication can be used. For example, the medication can be a medication for treating infestation by fleas.

In another aspect, provided herein is a kit or package, which kit or package comprises the above shampoo, and an instruction for washing a subject's hair using the shampoo.

### C. Uses of the Shampoos

In still another aspect, provided herein is a method for washing a subject's hair, which method comprises contacting a subject's hair with the above shampoo.

The present methods can be used for washing hair of any suitable subject. For example, the present methods can be used for washing hair of a human, e.g., a man, a woman, an old person, a middle aged person, a young person, a teenager, a child, a baby, an infant, etc. In some embodiments, the present methods can be used for washing hair of a baby or a child. In some embodiments, the present methods can be used for washing hair of a human that has a skin condition that affects the scalp of the human. The skin condition can be Dandruff or Seborrhoeic dermatitis (or seborrhoea). In another example, the present methods can be used for washing hair of an animal.

### D. Exemplary Embodiments

In some embodiments, provided herein is a personal hygiene cleaning liquid composition, which contains 0% to 12% of mild surfactant, 40% to 60% of one or more humectants and triple distilled water, which is characterized by the surface tension of the aqueous solution of the product is higher than 35 mN/m (the surface tension of pure water is 72 mN/m at 25° C.). In some embodiments, this composition has self-preservation properties.

In some embodiments, provided herein is a personal hygiene cleaning liquid composition, characterized in that: the surface tension of shampoo diluted 80 times is higher than 35 mN/m, while the surface tensions of other traditional shampoos at the same concentration are less than 30 mN/m, and most of them are between 24˜28 mN/m. In some embodiments, the surface tension of the present invention composition diluted 80 times is 37.5 mN/m.

In some embodiments, the above personal hygiene cleaning liquid composition or the shampoo contains a polymer substance that can form gel with coating properties, with or without a mild surfactant, and contains a preservative system or self-preservation.

In some embodiments, in the above personal hygiene cleaning liquid composition or the shampoo, the substance used to cover the surfactant is agar but not limited to agar, including any natural polymer substance that can form a gel, such as carrageenan, curd lan, gellan gum, etc. The appearance of the product can be solid or can be flowing liquid (actually composed of very fine irregular solid gel).

In some embodiments, in the above personal hygiene cleaning liquid composition or the shampoo, the chemically synthesized surfactant is mild sodium lauroyl sarcosinate or other safer surfactants. The exemplary safety standard is the surface tension value at a dilution of 1:6000; the higher the surface tension, the safer it is. The solid content of the surfactant is less than or equal to 3.6%, that is to say the content of the raw material with 30% sodium lauroyl sarcosinate in the shampoo is equal to or less than 12%.

In some embodiments, in the above personal hygiene cleaning liquid composition or the shampoo, the surfactant-free shampoo refers to the absence of any surfactant, including the natural extraction, microbial fermentation and chemically synthesized surfactants.

In some embodiments, the exemplary shampoo may be a shampoo that does not contain surfactants and preservatives.

In some embodiments, the exemplary shampoo may be a shampoos containing surfactants but no preservatives.

In some embodiments, the exemplary shampoo solves or improves the hair problems through the following measures:
1. Control the surface tension of the Shampoo solution diluted 80 times above 35 mN/m. During the normal shampoo process, the amount of water needed to soak 1 g of hair is about 80˜120 g, that is to say, the dilution factor of the shampoo is 80-120 times.
2. No sterilization raw materials are used in shampoo to maintain the natural micro-ecology of the skin. If you have serious hair problems, please consult a doctor.
3. Use natural polymer gel to wrap the surfactant, thereby reducing the amount of surfactant, and thus reducing the damage of the shampoo to the scalp.
4. Natural polymer materials are used as thickeners. From a large number of natural polymers, agar, carrageenan, curd lan and gellan gum can be used as gelling agents to encapsulate surfactants, among which the best is agar.

In some embodiments, the effect of exemplary shampoo is to provide a safer and environmentally friendly shampoo preparation method. The exemplary shampoo is prepared with a relatively mild amino acid surfactant or without any surfactant, with or without preservatives, and has good anti-dandruff and anti-hair loss effects.

### E. Examples

### Example 1. Comparative Safety Test of an Exemplary Shampoo and Current Shampoos

In the actual shampoo process, to fully infiltrate 1 gram hair, the amount of water required is 80 to 100 grams. Therefore, it is presumed that the actual real-time concentration of shampoo during shampooing is 1:80. The surface tension of the 1:80 shampoo-water solution was tested.

Test method. The experimental environment is at a constant temperature of 25° C., and the shampoo is diluted 80 times with triple distilled water. After completely dissolved, the supernatant is taken for testing. The surface tension is tested by JY-200C full-automatic interfacial tensiometer, referring to (GB5545 Surfactant Determination of surface tension by pulling up liquid film). The data was calibrated with a surface tension of triple distilled water of 72.0 mN/m. All formula ingredients of the exemplary shampoo of the present disclosure and other shampoos used in this comparative test are shown in Table 1A and 1B below. The comparative test data are shown in Tables 2 and 3 below.

TABLE 1A
Formula ingredients of the exemplary shampoo of the present disclosure

| | Brands and | |
|---|---|---|
| No. | its specific name | Ingredient |
| The | Moxi | AQUA, GLYCERIN, AGAR, SODIUM LAUROYL SARCOSINATE, CURD LAN, |
| Exemplary | | CARRAGEENAN, GELLAN GUM, ZEA MAYS (CORN) STARCH, MARIS LIMUS, |
| Shampoo | | METHYLCHLOROISOTHIAZOLINONE, METHYLISOTHIAZOLINONE |
| SH1 | Head & shoulders | AQUA, SODIUM LAURETH SULFATE, SODIUM LAURYL SULFATE, COCAMIDE |
| | Men's | MEA, ZINC CARBONATE, GLYCOL DISTEARATE, SODIUM CHLORIDE, |
| | anti-dandruff shampoo | FRAGRANCE, ZINC PYRITHIONE, SODIUM XYLENESULFONATE, DIMETHICONE, |
| | | CETYL ALCOHOL, HYDROCHLORIC ACID, GUAR HYDROXYPROPYLTRIMONIUM |
| | | CHLORIDE, MAGNESIUM SULFATE, SODIUM BENZOATE, MAGNESIUM |
| | | CARBONATE HYDROXIDE, BENZYL ALCOHOL, PROPANEDIOL, Citron Fruit Extract, |
| | | CI19140, METHYLCHLOROISOTHIAZOLINONE, METHYLISOTHIAZOLINONE, CI |
| | | 42090 |
| SH2 | Adolf | AQUA, SODIUM LAURETH SULFATE, COCAMIDOPROPYL BETAINE, DECYL |
| | Anti-dandruff shampoo | GLUCOSIDE, DIMETHICONE, POLYQUATERNIUM-7, AMMONIUM LAURYL |
| | | SULFATE, AMODIMETHICONE, COCAMIDE MEA, COCAMIDE DEA, GUAR |
| | | HYDROXYPROPYLTRIMONIUM CHLORIDE, ZINC PYRITHIONE, ACRYLATES/C10- |
| | | 30 ALKYL ACRYLATE CROSSPOLYMER, HYDROLYZED COLLAGEN PG-PROPYL |
| | | METHYLSILANEDIOL, SODIUM LAUROYL HYDROLYZED SILK, |
| | | TRICETYLMONIUM CHLORIDE, TRIDECETH-12, METHYLPARABEN, DMDM |
| | | HYDANTOIN, BEHENTRIMONIUM CHLORIDE, ETHYLPARABEN, ETIDRONIC ACID, |
| | | ZINC CHLORIDE, ROSA RUGOSA FLOWER OIL, FRAGRANCE, |
| | | METHYLISOTHIAZOLINONE, METHYLCHLOROISOTHIAZOLINONE, MAGNESIUM |
| | | NITRATE, MAGNESIUM CHLORIDE |
| SH3 | Clear | AQUA, SODIUM LAURETH SULFATE, COCAMIDOPROPYL BETAINE, |
| | Men's | DIMETHICONOL, ZINC PYRITHIONE, PROPANEDIO, ALKYL DIMETHICONE, GUAR |
| | anti-dandruff shampoo | HYDROXYPROPYLTRIMONIUM CHLORIDE, LYSINE HCL, GLYCERIN, MENTHOL, |
| | | CLIMBAZOLE, PANAX GINSENG ROOT EXTRACT, MELALEUCA ALTERNIFOLIA |
| | | (TEA TREE) LEAF EXTRACT, MENTHA PIPERITA (PEPPERMINT) LEAF EXTRACT, |
| | | PANTHENOL, TOCOPHERYL ACETATE, HELIANTHUS ANNUUS (SUNFLOWER) |
| | | SEED OIL, ZINC SULFATE, PYRIDOXINE HCL, SODIUM ASCORBYL PHOSPHATE, |
| | | FRAGRANCE, PPG-7, CARBOMER, CITRIC ACID, SODIUM HYDROXIDE, SODIUM |
| | | CHLORIDE, PHENOXYETHANOL, SODIUM SALICYLATE, SODIUM BENZOATE, CI |
| | | 42090, CI 47005 |
| SH4 | BAWANG | AQUA, SODIUM LAURETH SULFATE, SODIUM LAURYL SULFATE, |
| | DARKENING & | COCAMIDOPROPYL BETAINE, COCAMIDE DEA, ALKYL METHICONE, |
| | STRENGTHENING | STEARAMIDOPROPYL DIMETHYLAMINE, POLYQUATERNIUM-7, PANTHENOL, |
| | SHAMPOO | DISODIUM EDTA, CITRIC ACID, POLYQUATERNIUM-10, DMDM HYDANTOIN, |
| | | FRAGRANCE, PLATYCLADUS ORIENTALIS LEAF EXTRACT, POLYGONUM |
| | | MULTIFLORUM ROOT EXTRACT, CYNAMCHUM AURICULATUM ROOT EXTRACT, |
| | | SESAMUM INDICUM (SESAME) SEED EXTRACT, PAULOWNIA TOMENTOSA LEAF |
| | | EXTRACT, METHYLCHLOROISOTHIAZOLINONE, METHYLISOTHIAZOLINONE, |
| | | MAGNESIUM NITRATE, MAGNESIUM CHLORIDE, CARAMEL |
| SH5 | BEIJING | AQUA, AMMONIUM LAURETH SULFATE, PLATYCLADUS ORIENTALIS LEAF |
| | TONG RENTANG | EXTRACT, AMMONIUM LAURYL SULFATE, ZINGIBER OFFICINALE (GINGER) |
| | RESISTANCE | ROOT EXTRACT, ALKYL DIMETHICONE, SESAMUM INDICUM (SESAME) SEED |
| | SHAMPOO | EXTRACT, DISODIUM LAURETH SULFOSUCCINATE, GLYCOL DISTEARATE, |
| | | CETEARYL ALCOHOL, SODIUM CHLORIDE, POLYQUATERNIUM-10, SODIUM |
| | | CITRATE, DIAZOLIDINYL UREA, FRAGRANCE, CITRIC ACID, HYDROLYZED |
| | | WHEAT PROTEIN |
| SH6 | Little Dream Garden | AQUA, SODIUM LAURETH SULFATE, COCAMIDOPROPYL BETAINE, GLYCOL |
| | Amino acid oil control | STEARATE, SODIUM LAURYL SULFATE, COCAMIDE DEA, COCAMIDE MEA, |
| | shampoo | SODIUM COCOYL SARCOSINATE, SOPHORA ANGUSTIFOLIA ROOT EXTRACT, |
| | | LACTOBACILLUS/RICE FERMENT, ZINGIBER OFFICINALE EXTRACT, |
| | | ROSMARINUS OFFICINALIS (ROSEMARY) LEAF OIL, POLYGONUM MULTIFLORUM |
| | | EXTRACT, CAFFEINE, CETRIMONIUM CHLORIDE, GUAR |
| | | HYDROXYPROPYLTRIMONIUM CHLORIDE, PIROCTONE OLAMINE, SODIUM |
| | | CHLORIDE, POLYQUATERNIUM-7, DMDM HYDANTOIN, ACRYLATES/C10-30 |
| | | ALKYL ACRYLATE CROSSPOLYMER, POLYQUATERNIUM-10, ALLANTOIN, |
| | | CLIMBAZOLE, ZINC CHLORIDE, POTASSIUM HYDROXIDE, DISODIUM EDTA, |
| | | METHYLISOTHIAZOLINONE, METHYLCHLOROISOTHIAZOLINONE, MAGNESIUM |
| | | NITRATE, MAGNESIUM CHLORIDE, SALICYLIC ACID, DEXTRIN, NIACINAMIDE, |
| | | PEG-14M, CI 19140, CI 16255, FRAGRANCE |
| SH7 | PENTENE | AQUA, SODIUM LAURETH SULFATE, ALKYL DIMETHICONE, COCAMIDOPROPYL |
| | SILKY SMOOTH | BETAINE, SODIUM CITRATE, SODIUM XYLENESULFONATE, GLYCOL |
| | SHAMPOO | DISTEARATE, SODIUM CHLORIDE, CITRIC ACID, FRAGRANCE, STEARYL |
| | | ALCOHOL, CETYL ALCOHOL, GUAR HYDROXYPROPYLTRIMONIUM CHLORIDE, |
| | | SODIUM BENZOATE, TETRASODIUM EDTA, PANTHENOL, PANTHENYL ETHYL |
| | | ETHER, HYDROCHLORIC ACID, HISTIDINE, METHYLCHLOROISOTHIAZOLINONE, |
| | | CI 19140, METHYLISOTHIAZOLINONE, CI 17200 |

TABLE 1B
Formula ingredients of the exemplary shampoo of the present disclosure

| | Brands and | |
|---|---|---|
| No. | its specific name | Ingredient |
| The | Moxi | AQUA, GLYCERIN, AGAR, SODIUM LAUROYL SARCOSINATE, CURD LAN, |
| Exemplary | | CARRAGEENAN, GELLAN GUM, ZEA MAYS (CORN) STARCH, MARIS LIMUS, |
| Shampoo | | METHYLCHLOROISOTHIAZOLINONE, METHYLISOTHIAZOLINONE |
| SH-8 | Johnson | AQUA, PEG-80 SORBITAN LAURATE, COCAMIDOPROPYL BETAINE, POLYSORBATE 20, |
| | baby wash | SODIUM LAURETH SULFATE, SODIUM LAUROAMPHOACETATE, CITRIC ACID, SODIUM |
| | | BENZOATE, TETRASODIUM EDTA, p-ANISIC ACID, FRAGRANCE |
| SH-9 | Frog prince | AQUA, SODIUM LAURETH SULFATE, SODIUM LAUROAMPHOACETATE, DISODIUM |
| | Baby's shampoo & | LAURYL SULFOSUCCINATE, COCAMIDOPROPYL BETAINE, ACRYLATES COPOLYMER, |
| | baby wash | SODIUM CHLORIDE, COCAMIDE DEA, SODIUM STYRENE/ACRYLATES COPOLYMER, |
| | | FRAGARIA CHILOENSIS ANANASSA EXTRACT, MALUS PUMILA EXTRACT, 3-o-ETHYL |
| | | ASCORBIC ACID, PHENOXYETHANOL, CITRIC ACID, FRAGRANCE, |
| | | METHYLISOTHIAZOLINONE |
| SH-10 | Baby elephant | AQUA, PEG-80 SORBITAN LAURATE, SODIUM LAURETH SULFATE, COCAMIDOPROPYL |
| | Baby's shampoo & | BETAINE, PEG-150 DISTEARATE, BUTYLENE GLYCOL, SODIUM |
| | baby wash | COCOAMPHOACETATE, OLEA EUROPAEA (OLIVE) FRUIT OIL, CITRUS PARADISI |
| | | (GRAPEFRUIT) FRUIT EXTRACT, CITRIC ACID, FRAGRANCE, PHENOXYETHANOL, |
| | | DISODIUM EDTA, ETHYLHEXYLGLYCERIN, SODIUM BENZOATE |
| SH-11 | Good boy | AQUA, COCAMIDOPROPYL BETAINE, LAURAMIDOPROPYL HYDROXYSULTAINE, |
| | Baby olive hair and | SODIUM LAUROYL SARCOSINATE, DECYL GLUCOSIDE, GLYCERYL LAURATE, |
| | body wash | ACRYLATES/CETETH-20 METHACRYLATE COPOLYMER, COCAMIDE METHYL MEA, |
| | | DIMER DILINOLEAMIDOPROPYL PG-DIMONIUM CHLORIDE PHOSPHATE, OLEA |
| | | EUROPAEA EXTRACT, PORTULACA OLERACEA EXTRACT, HYDROLYZED WHEAT |
| | | PROTEIN, FRAGRANCE, SODIUM HYDROXIDE, METHYLISOTHIAZOLINONE |
| SH-12 | Missoue | AQUA, COCAMIDOPROPYL BETAINE, SODIUM COCOYL GLUTAMATE, LAURYL |
| | Top To Toe for baby | GLUCOSIDE, BUTYLENE GLYCOL, SODIUM LAUROAMPHOACETATE, |
| | strawberry | PANTHENOL, CITRIC ACID, ALOE BARBADENSIS LEAF EXTRACT, FRAGRANCE, |
| | | METHYLPARABEN, HYDROXYPROPYL GUAR HYDROXYPROPYLTRIMONIUM |
| | | CHLORIDE, DISODIUM EDTA |
| SH-13 | Babyganics | AQUA, SODIUM LAUROY METHYL ISETHIONATE, DISODIUM COCOAMPHODIACTATE, |
| | Bubble bath | COCO-GLUCOSIDE, LAURYL GLUCOSIDE, CAPRYL/CAPRAMIDOPROPYL BETAINE, |
| | | SOLANUM LYCOPERSICUM(TOMATO) SEED OIL, HELIANTHUS |
| | | ANNUUS(SUNFLOWER)SEED OIL, VACCININIUM MACROCARPON(CRANBERRY)SEED |
| | | OIL, NIGELLA SATIVA(BLACK CUMIN) SEED OIL, RUBUS IDAEUS (RASPBERRY) SEED |
| | | OIL, ALOE BARBADENSIS LEAF JUICE, CALENDULA OFFICINALIS FLOWER EXTRACT, |
| | | CHAMOMILLA RECUTITA (MATRICARIA) FLOWER EXTRACT, OLEA EUROPAEA |
| | | (OLIVE) LEAF EXTRACT, GLYCERIN, CITRIC ACID, TRISODIUM ETHYLENEDIAMINE |
| | | DISUCCINATE, PHENOXYETHANOL, ETHYLHEXYLGLYCERIN, SODIUM BENZOATE |
| SH-14 | Pigeon | AQUA, GLYCERIN, LAURAMIDOPROPYL BETAINE, PEG-20, SODIUM |
| | Baby soap | METHYLTAURATE, TEA-LAUROYL METHYLAMINOPROPIONATE, CERAMIDE 2, |
| | | ISOSTEARIC ACID, DIPOTASSIUM GLYCYRRHIZATE, PPG-4-CETETH-20, PDG, ETHYL |
| | | LAUROYL ARGINATE, LAURIC ACID, CITRIC ACID, SODIUM CITRATE, |
| | | PHENOXYETHANOL, ETHYLHEXYLGLYCERIN |
| SH-15 | Burt's bees baby | AQUA, CAPRYLYL/CAPRYL GLUCOSIDE, SODIUM LAUROYL GLUTAMATE, DISODIUM |
| | Shampoo & wash | COCOYL GLUTAMATE, ORYZA SATIVA(RICE)EXTRACT, SODIUM COCOYL |
| | | HYDROLYZED SOYPROTEIN, ORIGANUM VULGARE LEAF OIL, THYMUS |
| | | VULGARIS(TIIYME)OIL, XANTIIAN GUM. LACTIC ACID, LAURIC ACID, LAURYL |
| | | LACTYL LACTATE, SODIUM CHLORIDE, SODIUM COCOYL GLUTAMATE, SODIUM |
| | | LAUROYL LACTYLATE, POTASSIUM SORBATE, PHENOXYETHANOL |
| SH-16 | Pororo | AQUA, DISODIUM COCOAMPHODIACETATE, SODIUM CHLORIDE, HEXYLENE |
| | Marine 3 in 1 | GLYCOL, COCOGLYCERIDES, POTASSIUM COCOATE, COCAMIDOPROPYL BETAINE, |
| | | GLYCERIN, DISODIUM COCOYL GLUTAMATE, SODIUM PCA, LACTIC ACID, SODIUM |
| | | COCO PG-DIMONIUM CHLORIDE PHOSPHATE, 1,2-HEXANEDIOL, HYDROLYZED |
| | | WHEAT PROTEIN, FRAGRANCE, TETRASODIUM EDTA, PEG-120 METHYL GLUCOSE |
| | | DIOLEATE, METHYL GLUCETH-20, PANTHENOL, POLYGLYCERYL-10 LAURATE, |
| | | ETHYLHEXYLGLYCERIN, GLYCERYL CAPRYLATE, BUTYLENE GLYCOL, PERILLA |
| | | OCYMOIDES LEAF EXTRACT, CHAMOMILLA RECUTITA (MATRICARIA) EXTRACT, |
| | | GLYCYRRHIZA GLABRA EXTRACT, CENTELLA ASIATICA EXTRACT, ALTHAEA ROSEA |
| | | FLOWER EXTRACT, TARAXACUM OFFICINALE (DANDELION) LEAF EXTRACT, PANAX |
| | | NOTOGINSENG ROOT EXTRACT, SAPONARIA OFFICINALIS EXTRACT, BUPLEURUM |
| | | CHINENSIS EXTRACT, SALICORNIA HERBACEA EXTRACT, CHLORELLA MINUTISSIMA |
| | | EXTRACT, PORPHYRA YEZOENSIS EXTRACT |
| SH-17 | Arau | AQUA, SEKKEN SOJI-K, GLYCERIN, CITRIC ACID, CITRUS AURANTIFOLIA (LIME) OIL, |
| | Foam body soap | LAVANDULA ANGUSTIFOLIA (LAVENDER) OIL, BUTYLENE GLYCOL, PERILLA |
| | | OCYMOIDES LEAF EXTRACT, ROSMARINUS OFFICINALIS (ROSEMARY) LEAF |
| | | EXTRACT |
| SH-18 | Aveeno | AQUA, COCAMIDOPROPYL BETAINE, SODIUM LAUROMPHOACETATE, GLYCERIN, |
| | SCENTED | COCO-GLUCOSIDE, SODIUM CHLORIDE, CITRIC ACID, ACRYLATES/C10-30 ALKYL |
| | Wash & Shampoo | ACRYLATE CROSSPOLYMER, SODIUM BENZOATE, GLYCERYL OLEATE, SODIUM |
| | | HYDROXIDE, FRAGRANCE, POLYQUATERNIUM-10, GUAR |
| | LIGHTLY | HYDROXYPROPYLTRIMONIUM CHLORIDE, BUTYLENE GLYCOL, AVENA SATIVA(OAT) |
| | | KERNEL EXTRACT |
| SH19 | Sebamed | AQUA, DECYL GLUCOSIDE, DISODIUM LAURETH SULFOSUCCINATE, LAURETH-6 |
| | BABY & KIDS | CARBOXYLIC ACID, PANTHENOL, GLYCERYL LAURATE, TOCOPHERYL ACETATE, |
| | WASCHLOTION | MAGNESIUM SULFATE, LACTIC ACID, PEG/PPG-120/10, TRIMETHYLOLPROPANE |
| | HAUT & HAAR | TRIOLEATE, LAURETH-2, PEG-150 DISTEARATE, PARFUM, SODIUM HYDROXIDE, |
| | | ASCOPALMITATE, BENZYL ALCOHOL, PHENOXYETHANOL, SODIUM BENZOATE |
| SH20 | EARTH MAMA | AQUA, ORGANIC POTASSIUM COCOATE, ORGANIC POTASSIUM OLIVATE, ALOE |
| | Angel baby | BARBADENSIS(ORGANICALOE) LEAF JUICE, VANILLA PLANIFOLIA(ORGANIC |
| | baby wash & shampoo | VANILLA) FRUIT EXTRACT, CITRUS SINENSIS(ORGANIC SWEET ORANGE), KOSHER |
| | | VEGETABLE GLYCERIN, POTASSIUM CRTRATE, BUTYROSPERMUM PARKII (ORGANIC |
| | | SHEA) BUTTER, CALENDULA OFFICINALIS(ORGANIC CALENDULA) FLOWER |
| | | EXTRACT |
| SH21 | Ecostore | AQUA, DISODIUM COCOYL GLUTAMATE, SODIUM LAUROAMPHOACETATE, |
| | Safer for you | GLYCERIN, PANTHENOL, COCO-GLUCOSIDE, GLYCERYL OLEATE, BENZYL ALCOHOL, |
| | Foam to the max | DEHYDROACETIC ACID, CITRIC ACID, PARFUM, CITRAL LIMONENE |
| SH22 | Goongbe | AQUA, LAURYL GLUCOSIDE, DISODIUM COCOAMPHODIACETATE, COCO-BETAINE, |
| | Shampoo & bath | POTASSIUM COCOYL GLUTAMATE, COCO-GLUCOSIDE, SODIUM CHLORIDE, MORUS |
| | mild formula | ALBA LEAF EXTRACT, SALIX ALBA (WILLOW) BARK EXTRACT, SOPHORA JAPONICA |
| | inspired by korean | ROOT EXTRACT, PRUNUS PERSICA (PEACH) LEAF EXTRACT, PRUNUS MUME FRUIT |
| | wisdom & pure | EXTRACT, PHASEOLUS ANGULARIS SEED EXTRACT, AVENA SATIVA (OAT) KERNEL |
| | nature | OIL, QUILLAJA SAPONARIA BARK EXTRACT, GLYCERYL OLEATE, CAPRYLYL |
| | | GLYCOL, HEXYLENE GLYCOL, SODIUM COCOYL GLUTAMATE, ACRYLATES/C10-30 |
| | | ALKYL ACRYLATE CROSSPOLYMER, CITRIC ACID, SODIUM COCOYL ISETHIONATE, |
| | | ARGININE, 1,2-HEXANEDIOL, BUTYLENE GLYCOL, METHYLPROPANEDIOL, |
| | | ETHYLHEXYLGLYCERIN, DISODIUM EDTA, FRAGRANCE |
| SH23 | Ever eden | AQUA, ALOE BARBADENSIS LEAF JUICE, DECYL GLUCOSIDE, DISODIUM COCOYL |
| | | GLUTAMATE, PYRUS MALUS (APPLE) FRUIT EXTRACT, GLYCERIN, COCO-GLUCOSIDE, |
| | | GLYCERYL OLEATE, SODIUM COCOYL GLUTAMATE, SODIUM COCOYL GLYCINATE, |
| | | ARGININE, SIMMONDSIA CHINENSIS (JOJOBA) SEED OIL, PERSEA GRATISSIMA |
| | | (AVOCADO) OIL, HELIANTHUS ANNUUS (SUNFLOWER) SEED OIL, NIGELLA SATIVA |
| | | SEED OIL, CALENDULA OFFICINALIS FLOWER EXTRACT, CHAMOMILLA RECUTITA |
| | | (MATRICARIA) FLOWER EXTRACT, TERMINALIA FERDINANDIANA FRUIT EXTRACT, |
| | | GUAR HYDROXYPROPYLTRIMONIUM CHLORIDE, POTASSIUM SORBATE, SODIUM |
| | | PHYTATE, SODIUM BENZOATE, CITRIC ACID |
| SH24 | Calif RNIA | WATER, DECYL GLUCOSIDE, LAURY GLUCOSIDE, QUILLAJA SAPONARIA |
| | BABY | BARK EXTRACT, VEGETABLE GLYCERINE USP, CALENDULA OFFICINALIS |
| | SHAMPOO & | FLOWER EXTRACT(CALENDULA), VIOLA TRICOLOR EXTRACT(PANSY), |
| | BODY WASH | YUCCA SCHIDIGERA ROOT EXTRACT, ALOE BARBADENSIS LEAF JUICE, |
| | | SIMMONDSIA CHINENSIS (JOJOBA) SEED OIL, HYDROLYZED QUINOA, |
| | | XANTHAN GUM, PANTHENOL, PHYTIC ACID, GLUCONOLACTONE, SODIUM |
| | | BENZOATE |
| SH25 | Mustela | AQUA, COCAMIDOPROPYL BETAINE, SODIUM MYRETH SULFATE, PEG-7 GLYCERYL |
| | gentle cleansing | COCOATE, COCO-GLUCOSIDE, PEG-150 DISTEARATE, GLYCERYL CAPRYLATE, |
| | gel | FRAGRANCE, GLYCOL DISTEARATE, CITRIC ACID, PANTHENOL, POTASSIUM |
| | hair and body | SORBATE, PERSEA GRATISSIMA (AVOCADO) FRUIT EXTRACT |
| | cleans and softens | |
| SH26 | Jubilee | AQUA, SODIUM LAURETH SULFATE, COCAMIDOPROPYL BETAINE, SODIUM |
| | Baby ⁺2 | CHLORIDE, GLYCERIN, HEXYLENE GLYCOL, DISODIUM COCOAMPHODIACETATE, |
| | chamomile & | SODIUM COCOAMPHOACETATE, PARFUM, CITRIC ACID, PEG-150 DISTEARATE, |
| | honey | TETRASODIUM EDTA, MAGNESIUM NITRATE, METHYLCHLOROISOTHIAZOLINONE, |
| | | MAGNESIUM CHLORIDE, METHYLISOTHIAZOLINONE, HONEY EXTRACT, |
| | | CHAMOMILLA RECUTITA (MATRICARIA) FLOWER EXTRACT, SODIUM HYDROXIDE, CI |
| | | 42090, C119140, SODIUM BENZOATE, POTASSIUM SORBATE |
| SH27 | Babycocole | AQUA, SODIUM COCOAMPHOACETATE, SODIUM MYRETH SULFATE, |
| | Physiologic pH | COCAMIDOPROPYL BETAINE, PEG-120 METHYL GLUCOSE DIOLEATE, GLYCERIN, |
| | Delicate bath and | NELUMBO NUCIFERA ROOT EXTRACT, PEG-7 GLYCERYL COCOATE, PEG-200 |
| | shampoo | HYDROGENATED GLYCERYL PALMATE, PARFUM, C12-13 ALKYL LACTATE, |
| | | BISABOLOL, BETA-GLUCAN, PHENOXYETHANOL, SODIUM BENZOATE, CITRIC ACID |
| SH28 | Daramin | AQUA, COCO-GLUCOSIDE, SODIUM COCO-SULFATE, GLYCERYL CAPRYLATE, |
| | Girl | PROPYLENE GLYCOL, CITRIC ACID, SODIUM CHLORIDE, DISODIUM |
| | Shampoo & | COCOAMPHODIACETATE, GLYCERYL OLEATE, FRAGRANCE, SODIUM PCA, |
| | shower | GLYCERIN, HYDROLYZED WHEAT PROTEIN, GUAR HYDROXYPROPYLTRIMONIUM |
| | Fresh melon | CHLORIDE, PANTHENOL, SODIUM COCOYL APPLE AMINO ACIDS |
| SH29 | Sanosan | AQUA, SODIUM LAURETH SULFATE, GLYCERIN, COCAMIDOPROPYL BETAINE, |
| | Bath & Shampoo | SODIUM CHLORIDE, PEG-7 GLYCERYL COCOATE, PARFUM, CITRIC ACID, SODIUM |
| | Gel de banoy | BENZOATE, ALLANTOIN, POTASSIUM SORBATE, CARBOXYMETHYL |
| | champu | HYDROXYPROPYL GUAR, HYDROLYZED MILK PROTEIN, OLEA EUROPAEA (OLIVE) |
| | | LEAF EXTRACT |
| SH30 | Bubchen | AQUA, SODIUM LAURETH SULFATE, DISODIUM LAURETH SULFOSUCCINATE, |
| | SHAMPOO & | COCAMIDOPROPYL BETAINE, GLYCERIN, ALOE BARBADENSIS LEAF JUICE, CITRIC |
| | DUSCHGEL | ACID, HELIOTROPINE, HYDROLYZED WHEAT PROTEIN, PANTHENOL, PARFUM, PEG- |
| | 2 in 1 | 120 METHYL GLUCOSE DIOLEATE, PHENOXYISOPROPANOL, POLYQUATERNIUM-10, |
| | | SODIUM BENZOATE, SODIUM CHLORIDE, SODIUM CITRATE, SODIUM HYDROXIDE, |
| | | SODIUM SULFATE, TOCOPHEROL, CI 16035 |
| SH31 | Fish Price | AQUA, SODIUM MYRETH SULFATE, GLYCERIN, LAURYL GLUCOSIDE, |
| | kids care | COCAMIDOPROPYL BETAINE, PEG-120 METHYL GLUCOSE DIOLEATE, COCO- |
| | prebiotic emulsion | GLUCOSIDE, GLYCERYL OLEATE, SHEA BUTTER ETHYL ESTERS, PANTHENOL, |
| | for body and head | POLYQUATERNIUM-10, INULIN, ALPHA-GLUCAN OLIGOSACCHARIDE, CITRIC ACID, |
| | washing 2 in 1 | SODIUM LACTATE, SODIUM BENZOATE, POTASSIUM SORBATE, LACTIC ACID, |
| | | PARFUM, POLYSORBATE 20, SODIUM CHLORIDE |
| SH32 | FUNCARE | AQUA, SODIUM LAURET SULFATE, COCAMIDOPROPY BETAINE, COCAMIDOPROPYL |
| | 3 in 1 | HYDROXYSULTAINE, PEG/PPG-18/18 DIMETHICONE, PEG-150 DISTEARATE, PEG-80 |
| | body wash, | SORBOTAN LAURATE, POLYQUATERNIUM-10, GLYCOL DISTEARATE, STEARETH-4, |
| | Shampoo & | PARFUM, CITRIC ACID, TETRASODIUM EDTA, METHYL CHLOROISOTHIAZOLINONE, |
| | conditioner | METHYLISOTHIAZOLINONE, RED 40(CI 16035), RED 33(CI 17200) |

TABLE 2
The test results of surface tension of shampoos (Unit: mN/m)

| | The Exemplary | | | | | | | |
|---|---|---|---|---|---|---|---|---|
| | Shampoo | SH1 | SH2 | SH3 | SH4 | SH5 | SH6 | SH7 |
| Main | SODIUM | SODIUM | SODIUM | SODIUM | SODIUM | AMMONIUM | SODIUM | SODIUM |
| Surfactant | LAUROYL | LAURETH | LAURETH | LAURETH | LAURETH | LAURETH | LAURETH | LAURETH |
| | SARCOSINATE | SULFATE | SULFATE | SULFATE | SULFATE | SULFATE | SULFATE | SULFATE |
| Surface | 37.5 | 24.7 | 24.9 | 26.3 | 27.6 | 28.6 | 28.8 | 29.0 |
| Tension | | | | | | | | |
| 1:80 | | | | | | | | |

Note:
SHX are the code of shampoos, the product name and formula ingredient information are listed below.

TABLE 3
Comparison of surface tension between the shampoo of the present invention and domestic and overseas

| | | The Exemplary | | | | | |
|---|---|---|---|---|---|---|---|
| | | Shampoo | SH8 | SH9 | SH10 | SH11 | SH12 |
| Main Surfactant | | SODIUM | PEG-80 | SODIUM | PEG-80 | COCAMIDO- | COCAMIDO- |
| | | LAUROYL | SORBITAN | LAURETH | SORBITAN | PROPYL | PROPYL |
| | | SARCOSINATE | LAURATE | SULFATE | LAURATE | BETAINE | BETAINE |
| Surface | 1:80 | 37.5 | 23.9 | 25.8 | 27.6 | 28.3 | 30.7 |
| Tension | 1:100 | 38.6 | 24.2 | 26.3 | 27.9 | 28.5 | 31.2 |

| | | SH13 | SH14 | SH15 | SH16 | SH17 | SH18 |
|---|---|---|---|---|---|---|---|
| Main Surfactant | | SODIUM | LAURAMIDO- | CAPRYLYL/ | DISODIUM | SEKKEN | COCAMIDO- |
| | | LAUROY | PROPYL | CAPRYL | COCOAMPHODI- | SOJI-K | PROPYL |
| | | METHYL | BETAINE | GLUCOSIDE | ACETATE | | BETAINE |
| | | ISETHIONATE | | | | | |
| Surface | 1:80 | 24.3 | 24.7 | 24.9 | 25.3 | 25.3 | 25.5 |
| Tension | 1:100 | 24.8 | 25.2 | 25.1 | 25.5 | 25.6 | 25.9 |

| | | SH19 | SH20 | SH21 | SH22 | SH23 | SH24 | SH25 |
|---|---|---|---|---|---|---|---|---|
| Main Surfactant | | DECYL | ORGANIC | DISODIUM | LAURYL | DECYL | DECYL | COCAMIDO- |
| | | GLUCOSIDE | POTASSIUM | COCOYL | GLUCOSIDE | GLUCOSIDE | GLUCOSIDE | PROPYL |
| | | | COCOATE | GLUTAMATE | | | | BETAINE |
| Surface | 1:80 | 26.5 | 27.2 | 27.3 | 27.4 | 27.5 | 27.5 | 27.6 |
| Tension | 1:100 | 27.0 | 27.9 | 27.6 | 27.6 | 27.9 | 28.1 | 28.9 |

| | | SH26 | SH27 | SH28 | SH29 | SH30 | SH31 | SH32 |
|---|---|---|---|---|---|---|---|---|
| Main Surfactant | | SODIUM | SODIUM | COCO- | SODIUM | SODIUM | SODIUM | SODIUM |
| | | LAURETH | COCOAMPHO- | GLUCOSIDE | LAURETH | LAURETH | MYRETH | LAURET |
| | | SULFATE | ACETATE | | SULFATE | SULFATE, | SULFATE | SULFATE |
| Surface | 1:80 | 28.1 | 28.2 | 28.6 | 29.3 | 29.4 | 29.8 | 30.6 |
| Tension | 1:100 | 28.2 | 28.3 | 28.9 | 29.6 | 29.8 | 30.2 | 31.3 |

Note:
SHX are the code of children's shampoo;

As shown in above the test data, the surface tension of the traditional shampoo at real-time concentration is between 24 mN/m˜29 mN/m. Under the same concentration, the surface tension of the exemplary shampoo prepared by the present disclosure can reach 37.5 mN/m.

### Example 2. Fish Sensitivity Test

Fish are very sensitive to changes in the water environment, and fish acute toxicity tests are often used to measure water quality or chemical toxicity studies. In order to investigate the biological toxicity of shampoos, we conducted fish toxicity experiments.

Test method. The experimental environment is at a constant temperature of 25° C., and the shampoo is diluted 2000 times with triple distilled water. The fish acute toxicity test is carried out tin the aqueous solution diluted 6000 times with surfactant, referencing to (GB/T 21281-2007 fish acute toxicity test classification test method of hazardous chemicals). Specifically, 7 healthy small fish were taken into the shampoo-water solution and observe the survival status of the fish. All of the small fish died, then the experiment was over. According to the actual situation of the experiment, time is measured in days (d), hours (h) and minutes (min). The longest inspection period of the experiment is 7 days. Data records are shown in Table 4 below.

TABLE 4
Test results of fish acute toxicity of Shampoo

| | brand | | | | | | | |
|---|---|---|---|---|---|---|---|---|
| | | | | | | | Little | |
| | The Exemplary | Head | | | | Tong | Dream | |
| | Shampoo | shoulders | Adolf | Clear | BAWANG | Rentang | Garden | Pentene |
| survival | 7 d | 21 min | 26 min | 18 min | 34 min | 48 min | 25 min | 20 min |
| time | | | | | | | | |

As shown in above the test data, for traditional shampoo, in the aqueous solution diluted 2000 times, fish are poisoned by surfactants and other ingredients in the product, and all of the fish die within 1 hour, while the exemplary shampoo prepared by the present disclosure has no toxic effect on fish. The fish live healthy for 7 days or even longer.

Within the error range (excluding the inevitable fish individual differences and other uncertain components and contents in the shampoo), it can be considered that there is an opposite relationship between the fish survival time and the surface tension data. That is, the smaller the surface tension, the shorter the survival time of fish, showing that the shampoo is more toxic to fish. Based on this, we can predict the toxicity of shampoo to fish based on the surface tension data.

### Example 3. Further Comparative Safety Test of an Exemplary Shampoo and Current Shampoos

In order to further highlight the significant advantages of the safety of the shampoo of the present invention, we expanded the scope of research and examined the surface tension of children's shampoo and bath products at home and abroad. The test method is the same as in Example 1, and the data records are shown in Table 5 below.

TABLE 5
Comparison of surface tension between the shampoo
of the present invention and domestic and overseas

| | Brand | | | | | |
|---|---|---|---|---|---|---|
| | The Exemplary | | Frog | Baby | Good | |
| γ (mN/m) | Shampoo | Johnson | prince | elephant | boy | Missoue |
| 1:80 | 37.5 | 23.9 | 25.8 | 27.6 | 28.3 | 30.7 |
| 1:100 | 38.6 | 24.2 | 26.3 | 27.9 | 28.5 | 31.2 |

| | Brand | | | | | |
|---|---|---|---|---|---|---|
| | | | Burt's | | | |
| γ (mN/m) | Babyganics | Pigeon | bees | Pororo | arau | Aveeno |
| 1:80 | 24.3 | 24.7 | 24.9 | 25.3 | 25.3 | 25.5 |
| 1:100 | 24.8 | 25.2 | 25.1 | 25.5 | 25.6 | 25.9 |

Brand

| | | Angel | | | Ever | | |
|---|---|---|---|---|---|---|---|
| γ (mN/m) | Sebamed | baby | Ecostore | Goongbe | eden | Calif | Mustela |
| 1:80 | 26.5 | 27.2 | 27.3 | 27.4 | 27.5 | 27.5 | 27.6 |
| 1:100 | 27.0 | 27.9 | 27.6 | 27.6 | 27.9 | 28.1 | 28.9 |

| | Brand | | | | | | |
|---|---|---|---|---|---|---|---|
| | | | | | | Fish | |
| γ (mN/m) | Jubilee | babycocole | Daramin | Sanosan | Bubchen | Price | FUNCARE |
| 1:80 | 28.1 | 28.2 | 28.6 | 29.3 | 29.4 | 29.8 | 30.6 |
| 1:100 | 28.2 | 28.3 | 28.9 | 29.6 | 29.8 | 30.2 | 31.3 |

As shown in above the test data, Children's shampoos and bath products are mostly transparent pastes, which have better water solubility and more uniform and stable aqueous solutions. Their surface tension are generally low. The surface tension data of aqueous solutions diluted 80 times and 100 times are more than 90% below 30 mN/m.

Based on the above data analysis, it can be seen that within the scope of the investigation, whether it is an adult shampoo or a child shampoo, the surface tension of the actual use real-time concentration (1:80) is almost below 30 mN/m, while the surface tension of the exemplary shampoo prepared by the present disclosure is not less than 37.5 mN/m.

### Example 4. Safety Testing and Screening of Chemically Synthesized Surfactants

Almost all traditional shampoos contain chemically synthesized surfactants. The most common are anionic surfactants, such as sodium lauryl sulfate (SLS or K12), sodium laureth ether sulfate (AES or SLES), ammonium lauryl sulfate (K12-A), ammonium laureth ether sulfate (AES-A); followed by zwitterionic surfactants, such as cocamidopropyl betaine (CAB), Nonionic surfactant, such as Coconut oil fatty acid diethanolamide(6501).

We classify surfactants into three types: anionic surfactants, zwitterionic surfactants, and nonionic surfactants. Experimental studies on the surface tension and acute toxicity of fish were conducted on the above-mentioned main surfactants and other kinds of surfactants used in shampoo, bath and other care products.

Test method. The experimental environment is at a constant temperature of 25° C., the surfactants is diluted 200 and 6000 times with triple distilled water. The surface tension is tested by JY-200C full-automatic interfacial tensiometer, referring to (GB5545 Surfactant Determination of surface tension by pulling up liquid film). The fish acute toxicity test is carried out tin the aqueous solution diluted 6000 times with surfactant, referencing to (GB/T 21281-2007 fish acute toxicity test classification test method of hazardous chemicals). The data records of surface tension and fish acute toxicity experiment are shown in Tables 6-8.

1. Test data on anionic surfactant are shown in Table 6 below. In the scope of the anionic surfactant investigation, the aqueous solution diluted 6000 times still has a low surface tension. The fish acute toxicity experiments conducted at the same concentration have very short survival time. Especially K12 and AES, K12-A and AES-A, and AOS are widely used in traditional shampoos, the fish survive for no more than 30 minutes.

TABLE 6
Surface tension and experimental data
of acute toxicity of Anionic surfactant

| | | | Surface | | survival |
|---|---|---|---|---|---|
| | | | tension(mN/m) | | time |
| No. | Name of Anionic surfactants | | 1:200 | 1:6000 | (min.) |
| 1 | K12 | Sodium lauryl sulfate | 35.0 | 40.8 | 22 |
| 2 | K12-A | ammonium lauryl sulfate | 26.7 | 38.9 | 39 |
| 3 | AES | sodium laureth ether sulfate | 36.4 | 36.2 | 18 |
| 4 | AES-A | ammonium laureth ether | 34.9 | 32.9 | 23 |
| | | sulfate | | | |
| 5 | SCI-85 | Sodium Cocoyl Isethionate-85 | 30.6 | 42.8 | 24 |
| 6 | 4202 | Sodium lauroyl sarcosinate | 27.9 | 64.8 | >1440 |
| | | (30%) | | | |
| 7 | SAS | Sodium secondary alkyl | 31.3 | 41.5 | 41 |
| | | sulfonate | | | |
| 8 | AOS | Sodium α-alkenyl sulfonate | 31.8 | 41.8 | 20 |
| 9 | 1211 | Sodium dodecyl ether | 35.2 | 40.5 | 284 |
| | | phosphate | | | |

Within the scope of the investigation, the surface tension of sodium lauroyl sarcosinate (4202) at a concentration of 1:6000 is 64.8 mN/m, which is much higher than the surface tension of other surfactants; the acute toxicity experiment of fish conducted at this concentration, Fish can survive healthy for 24 hours. We extrapolate that sodium lauroyl sarcosinate (4202) is a mild surfactant.

2. Test data on zwitterionic surfactant are shown in Table 7 below. Among the zwitterionic surfactants, the most common cocamidopropyl betaine (CAB) has good cleaning and foaming properties, but it is indeed the most harmful among similar surfactants. In the acute toxicity experiment of fish conducted at 1:6000 concentration, the survival time of fish was only 27 minutes.

TABLE 7
Surface tension and experimental data of
acute toxicity of Zwitterionic surfactant

| | | | Surface | | |
|---|---|---|---|---|---|
| | Name of Zwitterionic | | tension (mN/m) | | survival |
| No. | surfactants | | 1:200 | 1:6000 | time (min.) |
| 1 | CAB | cocamidopropyl | 31.3 | 32.4 | 27 |
| | | betaine | | | |
| 2 | ASB | Sulfobetaine | 35.3 | 47.5 | 42 |
| 3 | MZL | Imidazoline | 29.1 | 45.5 | 134 |
| 4 | ZMLj | Imidazoline (import) | 28.5 | 31.3 | 64 |

3. Test data on nonionic surfactant are shown in Table 8 below. Within the scope of the investigation, non-ionic surfactants also have a great degree of harm. Even alkyl glycosides made from renewable plant materials can kill fish in about 30 minutes. The methyl glucose dioleate (DOE-120) has a relatively high surface tension; in fish acute toxicity experiments, fish can survive for 24 hours. However, DOE is only used as a thickener, not a foaming agent, because of its poor foaming properties and low cleaning ability.

TABLE 8
Surface tension and experimental data of
acute toxicity of Nonionic surfactant

| | | | Surface | | survival |
|---|---|---|---|---|---|
| | | | tension (mN/m) | | time |
| No. | Name of Nonionic surfactants | | 1:200 | 1:6000 | (min.) |
| 1 | AEO-3 | | 27.2 | 27.3 | 27 |
| 2 | AEO-7 | Fatty alcohol | 27.6 | 29.3 | 21 |
| | | polyoxyethylene ether | | | |
| 3 | AEO-9 | | 30.2 | 30.4 | 18 |
| 4 | 4501 | Lauramide propyl amine | 30.5 | 31.2 | 15 |
| | | oxide | | | |
| 5 | 6501 | Coconut oil fatty acid | 26.0 | 27.6 | 44 |
| | | diethanolamide | | | |
| 6 | APG1 | Alkyl glycoside (C8-C10) | 28.3 | 47.3 | 33 |
| 7 | APG2 | Alkyl glycoside (C12-C14) | 28.2 | 28.5 | 25 |
| 8 | APGj | Alkyl glycoside (import) | 28.1 | 28.6 | 25 |
| 9 | DOE-120 | Methyl glucose dioleate | 42.6 | 49.4 | >1440 |

In summary, the surfactant selected for the exemplary shampoo of the present disclosure is sodium lauroyl sarcosinate, and its maximum concentration is controlled to be 12% or less. It should be noted that the surfactant here refers to an aqueous solution containing 30% sodium lauroyl sarcosinate, and the solid raw material content of 100% sodium lauroyl sarcosinate in the actual shampoo is 3.6% or less.

### Example 5. Design of Exemplary Shampoos of the Present Disclosure

1: The establishment of safer shampoo standards. Based on the safety problems of the existing shampoos, the standards for safer shampoos are formulated as follows: At 25° C., dilute the shampoo with triple distilled water to 80 times, and measure the surface tension of the diluent. The surface tension equal to or higher than 35 is the standard for safer shampoo.

2: Selection and use of active substances. The active substance that can be used in the present invention is sodium lauroyl sarcosinate, or a safer active agent than sodium lauroyl sarcosinate, or no surfactant is used. Do not use more than one chemically synthesized surfactant, and do not use any other chemically synthesized hair conditioning agents such as fungicides.

3: Selection and use of natural polymer material as a thickener. Most of the natural thickeners come from plants, but also some extracts from animals and microorganisms and marine algae. So far, food thickeners used in the food industry in the world can be divided into four categories according to their sources: plants, marine algae, microorganisms and animals. Such as gelatin, sodium caseinate, gum arabic, locust bean gum, pectin, guar gum, tragacanth gum, konjac gum, round powder gum, wormwood gum, cassia gum, carrageenan, agar, alginic acid, Curd lan, xanthan gum, gellan gum, etc. are natural polymer materials commonly used.

Some of the above polymers used in shampoo may adhere to the hair and affect the smoothness and shine of the hair, as well as the fluffy effect of the hair. The invention selects those polymers with gel characteristics for thickening, which can minimize the deposition of polymers on the hair. Among them, agar, carrageenan, curd lan, gellan gum can meet the requirements of thickening shampoo and the requirement of no residual deposition.

4: Reduce the damage of surfactant to scalp by encapsulating the surfactant with gel. The surfactant is fixed in the gel, using the solidification properties of agar, carrageenan and curd lan, gellan gum. The gel is a kind of irregular particles with particle size from 150 um to 5 mm, preferably 300 um to 2 mm, depending on the production process and the formula of the shampoo. When the hardness of the particles is small enough, the product shows a flowing state and the product is composed of some small soft blocks. The product in this case is a soft gel and can be pressed out by the pump head.

The advantage of preparing gel shampoo is that it can reduce the contact of the shampoo with the scalp and thus reduce the damage to the scalp by wrapping surfactants in shampoo, even the amount of surfactant is equal. In addition, the physical adsorption of mineral raw materials (such as starch, cellulose, cyclodextrin, natural sea mud and diatomaceous earth) also have a good cleaning effect, which can reduce the amount of surfactant indirectly.

### Example 6. The Formula Design of Exemplary Safe Shampoos and Use Thereof

1. According to the above standards, we make the design plan as follow. The amount of sodium lauroyl sarcosinate (30% content) is equal to or less than 12% (that is, equivalent to solid content equal to or less than 3.6%). Agar, carrageenan, Curd lan and gellan gum are used as gelling agents to wrap the surfactant to form gel-like particles. Due to the difference in particle size and hardness, the product exhibit a flowing or solid appearance. Among the above gel raw materials, agar is preferably used as a gel of shampoo, and the usage amount is from 0.1% to 10%. Exemplary shampoo formulas as shown in Tables 9-13 below.

TABLES 9
Formula 1 (Flowing gel shampoo with surfactant
(with preservative system))

| | | | sodium lauroyl | | |
|---|---|---|---|---|---|
| | Glycerin | agar | sarcosinate (30%) | Casson | Water |
| XF1-03 | 12.5% | 3.0% | 3.0% | 0.05% | 81.5% |
| XF1-06 | 12.5% | 3.0% | 6.0% | 0.05% | 78.5% |
| XF1-09 | 12.5% | 3.0% | 9.0% | 0.05% | 75.5% |
| XF1-12 | 12.5% | 3.0% | 12.0% | 0.05% | 72.5% |

TABLES 10
Formula 2 (Flowing gel shampoo with
surfactant (no preservative system))

| | | | sodium lauroyl | |
|---|---|---|---|---|
| | Glycerin | agar | sarcosinate (30%) | Water |
| XF-12 | 30%-60% | 0.5%-0.9% | 7%-13% | 20%-35% |

TABLES 11
Formula 3 (Solid gel Shampoo containing particles
with surfactant (With preservative system))

| | | | sodium lauroyl | | |
|---|---|---|---|---|---|
| | Glycerin | agar | sarcosinate (30%) | Casson | Water |
| XF3-10 | 6%- | 1.8%- | 7%- | 0.05% | 45%- |
| | 12% | 2.6% | 11% | | 75% |

TABLES 12
Formula 4 (Solid gel Shampoo containing particles
with surfactant (no preservative system))

| | | | sodium lauroyl | |
|---|---|---|---|---|
| | Glycerin | agar | sarcosinate (30%) | Water |
| XF4-10 | 40%-60% | 1.1-1.5% | 7%-12% | 40%-70% |

TABLE 13
Formula 5 (Flowing gel shampoo without surfactant) (Add cellulose
and starch or mineral mud) (with preservative system)

| | Psyllium | Konjac | | | | corn | Red | | |
|---|---|---|---|---|---|---|---|---|---|
| | Husk Powder | Glucomannan | agar | Glycerin | Betaine | starch | mud | Casson | Water |
| XF5 | 0.2-0.6 | 0.6-1.2 | 0.2-05 | 6-10 | 8-12 | 14-20 | 2-5 | 0.05 | 45-70 |

2: Surface tension testing of some formulations. Test method. The experimental environment is at a constant temperature of 25° C., and the shampoo is diluted 80 times and 100 times with triple distilled water. After completely dissolved, the supernatant is taken for testing. The surface tension is tested by JY-200C full-automatic interfacial tensiometer, referring to (GB5545 Surfactant Determination of surface tension by pulling up liquid film). The data was calibrated with a surface tension of triple distilled water of 72.0 mN/m. Surface tension is marked with γ (mN/m). The test data are shown in Table 14 below.

TABLE 14

Shampoo

| γ (mN/m) | | XF1-3 | XF1-6 | XF1-9 | XF1-12 |
|---|---|---|---|---|---|
| surface | 1:80 | 49.5 | 40.2 | 39.7 | 37.5 |
| tension of | 1:100 | 53.1 | 44.6 | 43.6 | 38.6 |
| shampoo | | | | | |

It can be seen from the surface tension data above that all of the surface tension of the shampoo is higher than 37.5 mN/m in 1:80 dilution and higher than 38.6 mN/m in 1:100 dilution. The value of surface tension increases with decreasing surfactant content.

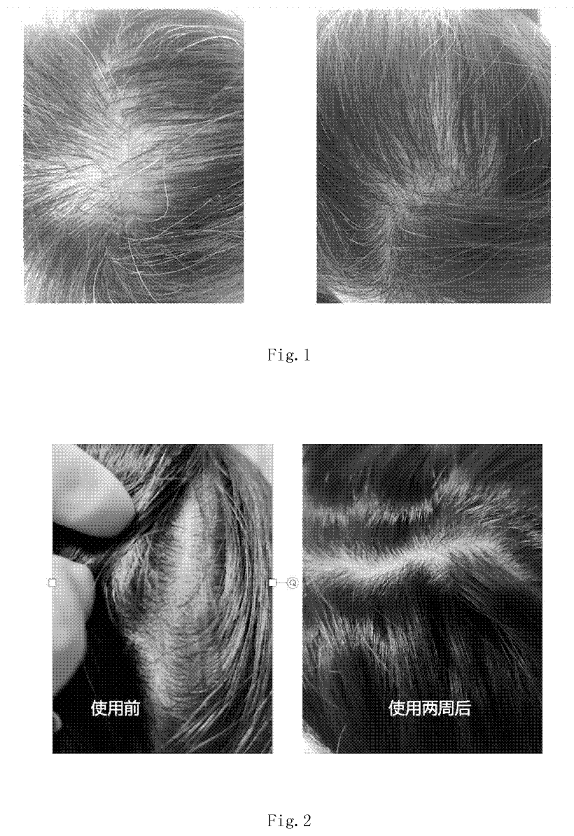

Some of the above exemplary shampoos were evaluated on various volunteers. In one study, XF1-12 was evaluated on Volunteer 1 (42 years old, female). As shown in FIG. 1, before using the shampoo of the present invention, volunteer 1 has a serious dandruff problem. After using this shampoo for 30 days, the dandruff problem was solved. And then after 60 days, new hair appears. The unexpected surprise is that the scalp has changed from an unhealthy red color to a normal color.

In another study, XF1-12 was evaluated on Volunteer 2 (9 years old, female). As shown in FIG. 2, after using shampoo XF1-12 for two weeks, the dandruff was completely removed; the hair remained healthy and normal for the next two months, and the dandruff problem did not rebound.

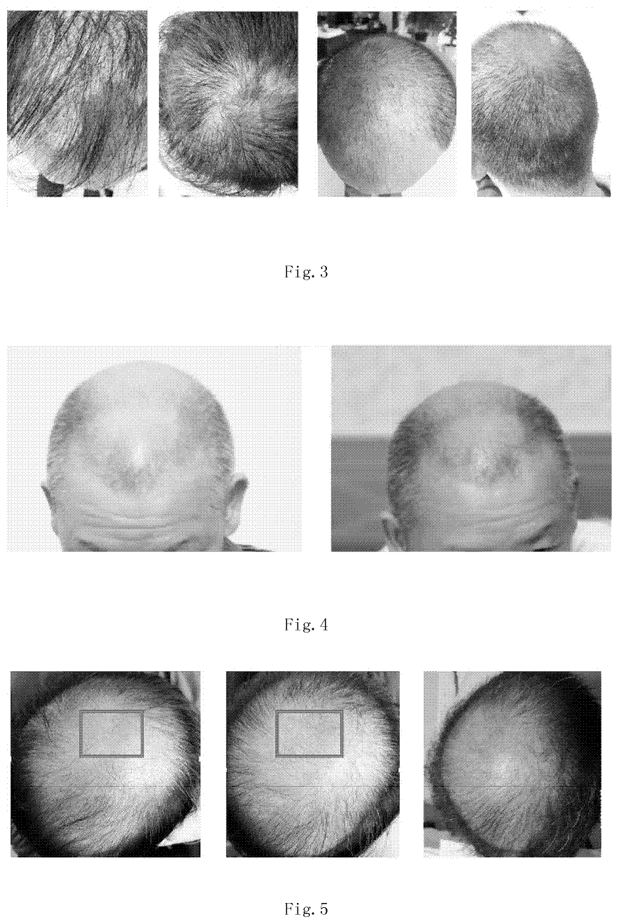

In still another study, XF1-6 was evaluated on Volunteer 3 (41 years old, male). As shown in FIG. 3, Volunteer 3 has severe dandruff, and the scalp is itchy and red, the hair is severely shed, and he starts to baldness. After trying many shampoos, it has not been solved, and the problem is getting worse. After using XF1-06 shampoo for one week, the scalp no longer itchy. After two months, there is no dandruff, the scalp returns to normal luster, and a lot of new hair grows.

In yet another study, XF1-6 was evaluated on Volunteer 5 (53 years old, male). As shown in FIG. 4, Volunteer 4's hair has begun to show signs of baldness due to severe hair loss, a lot of white hair, and acne on the scalp, and a burning sensation when touched. After using the shampoo for 12 weeks, the small acne on the head disappeared completely, the hair started to turn black, and new hair grew. Since using the shampoo for 10 months, his hair has become healthier and he has no more acne.

In yet another study, XF1-6 was evaluated on Volunteer 5 (48 years old, male). As shown in FIG. 5, Volunteer 5 has thin hair, baldness and greasy scalp. After using the shampoo, his scalp is no longer greasy. and a lot of new hair grows after using the shampoo 180 days.

Throughout the experience of the various volunteers mentioned above, the exemplary shampoos of the present disclosure greatly reduces the damage to the hair caused by a large amount of chemically synthesized surfactants. The exemplary shampoos of the present disclosure does not use any bactericide, and helps to restore the microecology of the scalp, eliminate damage from the source, restore the body's own repair ability, and thus solve or improve the various hair problems.

1. A shampoo, when diluted for at least about 80 times, the surface tension of the diluent from the shampoo, measured at an ambient temperature, is at about 35 mN/m or higher.

2. (canceled)

3. The shampoo of claim 1, when diluted for about 80 times, the surface tension of the diluent from the shampoo, measured at an ambient temperature, ranges from about 35 mN/m to about 68 mN/m.

4. (canceled)

5. The shampoo of claim 1, which does not comprise a surfactant.

6. The shampoo of claim 1, which comprises a surfactant.

7-8. (canceled)

9. The shampoo of claim 6, wherein the surfactant is sodium lauroyl sarcosinate, methyl glucose dioleate (DOE-120), ASB-14 (amidosulfobetaine-14), or Alkyl glycoside (C8-C10) (APG1).

10-13. (canceled)

14. The shampoo of claim 1, which further comprises a gel-forming natural polymer.

15-16. (canceled)

17. The shampoo of claim 14, wherein the gel formed by the natural polymer encapsulates the surfactant.

18-19. (canceled)

20. The shampoo of claim 14, wherein the natural polymer is selected from the group consisting of agar, carrageenan, curd lan, and gellan gum.

21. (canceled)

22. The shampoo of claim 1, which further comprises a humectant.

23-24. (canceled)

25. The shampoo of claim 1, which does not comprise a preservative.

26. The shampoo of claim 1, which further comprises a preservative.

27-28. (canceled)

29. The shampoo of claim 1, which further comprises an additional substance that is starch, cellulose, cyclodextrin, natural sea mud and/or diatomaceous earth.

30-31. (canceled)

32. The shampoo of claim 1, which further comprises water.

33. (canceled)

34. The shampoo of claim 32, wherein the level or concentration of the water ranges from 10% (w/w) to about 90% (w/w).

35. The shampoo of claim 1, which is configured as a solid, a semi-liquid, a flowing liquid, a viscos liquid, a gel, a flowing gel, a soft gel or a liquid.

36-50. (canceled)

51. The shampoo of claim 1, which is configured as a baby shampoo.

52. The shampoo of claim 1, which is configured as a shampoo for washing hair of a subject or a human with a skin condition that affects the scalp of the subject or human.

53-55. (canceled)

56. The shampoo of claim 1, which is configured as an animal shampoo.

57-58. (canceled)

59. A kit or package, which kit or package comprises a shampoo of claim 1, and an instruction for washing a subject's hair using the shampoo.

60. A method for washing a subject's hair, which method comprises contacting a subject's hair with a shampoo of claim 1.

61-65. (canceled)
